(19) United States
(12) Patent Application Publication
(10) Pub. No.: US 2021/0163605 A1
(43) Pub. Date: Jun. 3, 2021

(54) RAMUCIRUMAB FOR THE TREATMENT OF CANCERS IN PEDIATRIC PATIENTS

(71) Applicant: Eli Lilly and Company, Indianapolis, IN (US)

(72) Inventor: Heather Ann WASSERSTROM, Tenafly, NJ (US)

(21) Appl. No.: 17/045,246

(22) PCT Filed: Mar. 29, 2019
(86) PCT No.: PCT/US2019/024746

§ 371 Date: Oct. 5, 2020

Related U.S. Application Data
(60) Provisional application No. 62/653,662, filed on Apr. 6, 2018.

(51) Int. Cl. C07K 16/28; A61P 35/00

(52) U.S. Cl. CPC C07K 16/2863; A61K 2039/505; A61P 35/00

(57) ABSTRACT

The invention provides for methods, treatments, and uses for ramucirumab for the treatment of pediatric cancers.

The present invention relates to the field of cancer treatment. More specifically, the present invention relates to the use of ramucirumab for the treatment of cancers in pediatric patients.

Pediatric patients with solid tumors tend to have a poor prognosis when the patients display metastic or recurrent disease. While bevacizumab has been studied in pediatric tumors (Gururangan S, Fangusaro J, Poussaint T Y, et al: Efficacy of bevacizumab plus irinotecan in children with recurrent low-grade gliomas—a Pediatric Brain Tumor Consortium study, Neuro Oncol 16:310-7, 2014), ramucirumab may provide an efficacious alternative for said patients as it provides a distinct mechanism of action, in addition to its specificity, potency, and potential for combinations with other therapeutics.

As used herein, the term “about” means a deviation from a given value by no more or less than 10%. As a non-limiting example, when used in regards to weight, “about 100 mg” denotes a range from 90 mg (inclusive) to 110 mg (inclusive).

As used herein, the term “human VEGFR-2” refers to Human Vascular Endothelial Growth Factor Receptor 2 having the amino acid sequence of SEQ ID NO: 5. VEGFR-2 is also known as Fetal Liver Kinase-1 (FLK1), and Kinase Insert Domain Receptor (KDR).

As used herein, the term “human VEGF-D” refers to human vascular endothelial growth factor-D having the amino acid sequence of SEQ ID NO: 6.

Ramucirumab is a human IgG1 monoclonal antibody directed against human vascular endothelial growth factor receptor 2 (VEGFR-2). Ramucirumab and methods of making and using ramucirumab have been previously disclosed. Ramucirumab is approved by the United States Food and Drug Administration as a single agent or in combination with paclitaxel, for the treatment of advanced gastric or gastro-esophageal junction adenocarcinoma, with disease progression on or after prior fluoropyrimidine- or platinum-containing chemotherapy; in combination with docetaxel, for the treatment of metastatic non-small cell lung cancer with disease progression on or after platinum-based chemotherapy; and in combination with FOLFIRI (irinotecan, folinic acid, and 5-fluorouracil), for the treatment of metastatic colorectal cancer with disease progression on or after prior therapy with bevacizumab, oxaliplatin, and a fluoropyrimidine.

A non-limiting example of ramucirumab is CYRAIVIZA® and has the CAS registry number 947687-13-0. Ramucirumab is an anti-human VEGFR-2 antibody comprising two light chains, each of the light chains having the amino acid sequence of SEQ ID NO: 3, and two heavy chains, each of the heavy chains having the amino acid sequence of SEQ ID NO: 4. The light chain variable region of ramucirumab is that given in SEQ ID NO: 1. The heavy chain variable region of ramucirumab is that given in SEQ ID NO: 2.

As used herein, the terms “treating,” “treat,” or “treatment” refer to restraining, slowing, lessening, reducing, or reversing the progression or severity of an existing symptom, disorder, condition, or disease, or ameliorating clinical symptoms of a condition. Beneficial or desired clinical results include, but are not limited to, alleviation disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In some embodiments, the present invention can be used as a medicament.

As used herein, the term “cancer” refers to or describes the physiological condition in mammals that is typically characterized by unregulated cell growth. Included in this definition are benign and malignant cancers.

In the methods of the present invention, a therapeutically effective amount of an anti-VEGFR-2 antibody described herein is administered to a mammal or patient in need thereof. Additionally, the pharmaceutical compositions of the invention may include a therapeutically effective amount of an anti-VEGFR-2 antibody described herein.

A “therapeutically effective amount,” refers to an amount effective, at dosages and for periods of time necessary, to achieve the desired therapeutic result. A therapeutically effective amount can be readily determined by the attending diagnostician, as one skilled in the art, by the use of known techniques and by observing results obtained under analogous circumstances. In determining the therapeutically effective amount for a patient, a number of factors are considered by the attending diagnostician, including, but not limited to: the species of patient; its size, age, and general health; the specific disease or disorder involved; the target site; the degree of the severity of the disease or disorder; the response of the individual patient; the particular compound administered; the mode of administration; the bioavailability characteristics of the preparation administered; the dose regimen selected; the use of concomitant medication; other medications administered; and other relevant circumstances. A therapeutically effective amount is also one in which any toxic or detrimental effects of the antibody or antibody portion are outweighed by the therapeutically beneficial effects.

Generally, dosage regimens may be adjusted to provide the optimum desired response (e.g., a therapeutic response). In some embodiments, the dose for pediatric patients is from about 6 mg/kg to about 16 mg/kg, alternatively from about 6 mg/kg to about 12 mg/kg, alternatively from about 6 mg/kg to about 10mg/kg, alternatively from about 6 mg/kg to about 8 mg/kg, alternatively from about 8 mg/kg to about 16 mg/kg, alternatively from about 8 mg/kg to about 12 mg/kg, alternatively from alternatively from about 8 mg/kg to about 10 mg/kg, alternatively from about 10 mg/kg to about 16 mg/kg, alternatively from about 10 mg/kg to about 12 mg/kg, or alternatively from about 12 mg/kg to about 16 mg/kg. In some embodiments, the dose for pediatric patients is 6 mg/kg, 8 mg/kg, 10 mg/kg, 12 mg/kg, or 16 mg/kg. In some embodiments, the aforementioned doses are intravenously infused over 60 minutes, every 2 weeks.

In some instances, dosage levels below the lower limit of the aforesaid dosing for ramucirumab may be more than adequate, while in other cases larger doses may be employed with acceptable side effects, and therefore the above dosage amount is not intended to limit the scope of the invention in any way.

In the present invention, any suitable method or route can be used to administer an anti-VEGFR-2 antibody described herein, although intravenous (i.v.) administration is the preferred route. It should be emphasized, however, that the present invention is not limited to any particular method or route of administration.

The anti-human VEGFR-2 antibodies, including but not limited to ramucirumab, where used in a patient for the purpose of treatment, is preferably formulated as a pharmaceutical composition. Such pharmaceutical compositions and processes for preparing the same are well known in the art. See, e.g. Remington: The Science and Practice of Pharmacy (Gennaro A., et al., eds., 19th ed., Mack Publishing Co., 1995).

Unless indicated otherwise, the term “antibody” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes the amino acid sequences of CDRs and framework regions (FRs).

As used herein, the term “heavy chain variable region” “HCVR” refers to a portion of a heavy chain of an antibody molecule that includes the amino acid sequences of CDRs and FRs.

The antibodies described herein may readily be produced in mammalian cells, non-limiting examples of which includes CHO, NSO, HEK293 or COS cells. The host cells are cultured using techniques well known in the art. In this regard, an appropriate host cell can be either transiently or stably transfected with an expression system for secreting antibodies using an optimal predetermined HC:LC vector ratio or a single vector system encoding both HC (heavy chain) and LC (light chain). The vectors containing the polynucleotide sequences of interest (e.g., the polynucleotides encoding the polypeptides of the antibody and expression control sequences) can be transferred into the host cell by well-known methods, which may vary depending on the type of cellular host. Clarified media, into which the antibody has been secreted, may be purified using any of many commonly-used techniques. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, N.Y. (1994). In some examples, the medium may be conveniently applied to a column that has been equilibrated with a compatible buffer. The column may be washed to remove nonspecific binding components. The bound antibody may be eluted, for example, by pH gradient. Antibody fractions may be detected, such as by UV absorbance or SDS-PAGE, and then may be pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purity of the antibody after these chromatography steps is typically greater than 95%. The product may be immediately frozen at −70° C. or may be lyophilized.

The present disclosure provides a method of treating cancer in a human patient comprising administering a therapeutically effective amount of ramucirumab to the human patient in need thereof; wherein the human patient has been diagnosed with one of the following cancers: osteosarcoma, sarcoma, pheochromocytoma, paraganglioma, Wilms' tumor, hepatoblastoma, carcinoma NOS, synovial sarcoma, or desmoplastic small round cell tumor.

The present disclosure provides a method of treating cancer in a human patient comprising administering a therapeutically effective amount of ramucirumab to the human patient in need thereof; wherein the human patient has been diagnosed with one of the following cancers: osteosarcoma, sarcoma, pheochromocytoma, paraganglioma, Wilms' tumor, hepatoblastoma, carcinoma NOS, synovial sarcoma, or desmoplastic small round cell tumor; wherein the human patient is at least twelve months of age, but less than or equal to twenty one years of age.

The present disclosure provides a method of treating cancer in a human patient comprising administering a therapeutically effective amount of ramucirumab to the human patient in need thereof; wherein the human patient has been diagnosed with osteosarcoma. The present disclosure provides a method of treating cancer in a human patient comprising administering a therapeutically effective amount of ramucirumab to the human patient in need thereof; wherein the human patient has been diagnosed with sarcoma. The present disclosure provides a method of treating cancer in a human patient comprising administering a therapeutically effective amount of ramucirumab to the human patient in need thereof; wherein the human patient has been diagnosed with pheochromocytoma. The present disclosure provides a method of treating cancer in a human patient comprising administering a therapeutically effective amount of ramucirumab to the human patient in need thereof; wherein the human patient has been diagnosed with paraganglioma. The present disclosure provides a method of treating cancer in a human patient comprising administering a therapeutically effective amount of ramucirumab to the human patient in need thereof; wherein the human patient has been diagnosed with Wilms' tumor. The present disclosure provides a method of treating cancer in a human patient comprising administering a therapeutically effective amount of ramucirumab to the human patient in need thereof; wherein the human patient has been diagnosed with hepatoblastoma. The present disclosure provides a method of treating cancer in a human patient comprising administering a therapeutically effective amount of ramucirumab to the human patient in need thereof; wherein the human patient has been diagnosed with carcinoma NOS. The present disclosure provides a method of treating cancer in a human patient comprising administering a therapeutically effective amount of ramucirumab to the human patient in need thereof; wherein the human patient has been diagnosed with synovial sarcoma. The present disclosure provides a method of treating cancer in a human patient comprising administering a therapeutically effective amount of ramucirumab to the human patient in need thereof; wherein the human patient has been diagnosed with desmoplastic small round cell tumor.

In some examples, ramucirumab is administered intravenously every 2 weeks at a dose from about 6 mg/kg to about 16 mg/kg. In some examples, ramucirumab is administered intravenously every 2 weeks at a dose from 6 mg/kg to 16 mg/kg. In some examples, ramucirumab is administered intravenously every 2 weeks at a dose from about 8 mg/kg to about 12 mg/kg. In some examples, ramucirumab is administered intravenously every 2 weeks at a dose from 8 mg/kg to 12 mg/kg. In some examples, ramucirumab is administered intravenously every 2 weeks at a dose of about 6 mg/kg. In some examples, ramucirumab is administered intravenously every 2 weeks at a dose of about 8 mg/kg. In some examples, ramucirumab is administered intravenously every 2 weeks at a dose of about 10 mg/kg. In some examples, ramucirumab is administered intravenously every 2 weeks at a dose of about 12 mg/kg. In some examples, ramucirumab is administered intravenously every 2 weeks at a dose of about 16 mg/kg.

The present disclosure provides ramucirumab for use in the treatment of a human patient having osteosarcoma, sarcoma, pheochromocytoma, paraganglioma, Wilms' tumor, hepatoblastoma, carcinoma NOS, synovial sarcoma, or desmoplastic small round cell tumor. The present disclosure provides ramucirumab for use in the treatment of a human patient having osteosarcoma, sarcoma, pheochromocytoma, paraganglioma, Wilms' tumor, hepatoblastoma, carcinoma NOS, synovial sarcoma, or desmoplastic small round cell tumor; wherein the human patient is at least twelve months of age, but less than or equal to twenty one years of age.

The present disclosure provides ramucirumab for use in the treatment of a human patient having osteosarcoma. The present disclosure provides ramucirumab for use in the treatment of a human patient having sarcoma. The present disclosure provides ramucirumab for use in the treatment of a human patient having pheochromocytoma. The present disclosure provides ramucirumab for use in the treatment of a human patient having paraganglioma. The present disclosure provides ramucirumab for use in the treatment of a human patient having Wilms' tumor. The present disclosure provides ramucirumab for use in the treatment of a human patient having hepatoblastoma. The present disclosure provides ramucirumab for use in the treatment of a human patient having carcinoma NOS. The present disclosure provides ramucirumab for use in the treatment of a human patient having synovial sarcoma. The present disclosure provides ramucirumab for use in the treatment of a human patient having desmoplastic small round cell tumor.

The present disclosure provides a pharmaceutical composition comprising ramucirumab for use in the treatment of a human patient having osteosarcoma, sarcoma, pheochromocytoma, paraganglioma, Wilms' tumor, hepatoblastoma, carcinoma NOS, synovial sarcoma, or desmoplastic small round cell tumor. The present disclosure provides a pharmaceutical composition comprising ramucirumab for use in the treatment of a human patient having osteosarcoma, sarcoma, pheochromocytoma, paraganglioma, Wilms' tumor, hepatoblastoma, carcinoma NOS, synovial sarcoma, or desmoplastic small round cell tumor; wherein the human patient is at least twelve months of age, but less than or equal to twenty one years of age.

The present disclosure provides a pharmaceutical composition comprising ramucirumab for use in the treatment of a human patient having osteosarcoma. The present disclosure provides a pharmaceutical composition comprising ramucirumab for use in the treatment of a human patient having sarcoma. The present disclosure provides a pharmaceutical composition comprising ramucirumab for use in the treatment of a human patient having pheochromocytoma. The present disclosure provides a pharmaceutical composition comprising ramucirumab for use in the treatment of a human patient having paraganglioma. The present disclosure provides a pharmaceutical composition comprising ramucirumab for use in the treatment of a human patient having Wilms' tumor. The present disclosure provides a pharmaceutical composition comprising ramucirumab for use in the treatment of a human patient having hepatoblastoma. The present disclosure provides a pharmaceutical composition comprising ramucirumab for use in the treatment of a human patient having carcinoma NOS. The present disclosure provides a pharmaceutical composition comprising ramucirumab for use in the treatment of a human patient having synovial sarcoma. The present disclosure provides a pharmaceutical composition comprising ramucirumab for use in the treatment of a human patient having desmoplastic small round cell tumor.

Inclusion criteria for the study is as follows: patients must be ≥12 months and ≤21 years of age at the time of study enrollment. Patients with recurrent or refractory non-CNS solid tumors are eligible. Patients must have had histologic verification of malignancy at original diagnosis or relapse except patients with extra-cranial germ-cell tumors who have elevations of serum tumor markers including alpha-fetoprotein or beta-HCG. Patients in Part A cannot have CNS metastases. Patients with recurrent or refractory CNS tumors will be eligible and must have a histological verification of malignancy at original diagnosis or relapse except in patients with intrinsic brain stem tumors, optic pathway gliomas, or patients with CNS-germ cell tumors and elevations of CSF or serum tumor markers including alpha-fetoprotein or beta-HCG. Patient's current disease state must be one for which there is no known curative therapy or therapy proven to prolong survival with an acceptable quality of life. Performance Level: Karnofsky ≥50% for patients >16 years of age and Lansky ≥50 for patients ≤16 years of age. Neurologic deficits in patients with CNS tumors must have been relatively stable for at least 7 days prior to study enrollment. Patients who are unable to walk because of paralysis, but who are up in a wheelchair, will be considered ambulatory for the purpose of assessing the performance score. Patients must have fully recovered from the acute toxic effects of all prior anti-cancer therapy: a. Myelosuppressive chemotherapy: at least 21 days after the last dose of myelosuppressive chemotherapy (42 days if prior nitrosourea); b. hematopoietic growth factors: at least 14 days after the last dose of a long-acting growth factor (e.g. Neulasta) or 7 days for short-acting growth factor. For agents that have known adverse events occurring beyond 7 days after administration, this period must be extended beyond the time during which adverse events are known to occur. The duration of this interval must be discussed with the study chair; c. Biologic (anti-neoplastic agent): at least 7 days after the last dose of a biologic agent. For agents that have known adverse events occurring beyond 7 days after administration, this period must be extended beyond the time during which adverse events are known to occur. The duration of this interval must be discussed with the study chair; d. Immunotherapy: at least 42 days after the completion of any type of immunotherapy, e.g. tumor vaccines; e. Monoclonal antibodies: at least 3 half-lives of the antibody after the last dose of a monoclonal antibody. (See table on DVL homepage listing monoclonal antibody half-lives.); f XRT: At least 14 days after local palliative XRT (small port); At least 150 days must have elapsed if prior TBI, craniospinal XRT or if ≥50% radiation of pelvis; At least 42 days must have elapsed if other substantial BM radiation; g. Stem Cell Infusion without TBI: No evidence of active graft vs. host disease and at least 84 days must have elapsed after transplant or stem cell infusion; h. Patients must not have received prior exposure to ramucirumab. Patients must also have adequate bone marrow function, adequate renal function, adequate liver function, adequate cardiac function, adequate blood pressure control, adequate coagulation, and provide informed consent.

Exclusion criteria for the study is as follow: Pregnant or breast-feeding women will not be entered on this study because there is yet no available information regarding human fetal or teratogenic toxicities; patients under concomitant medications including corticosteroids, investigational drugs, anti-cancer agents, anti-GVHD agents post-transplant, anti-inflammatory agents, anti-platelet agents, anti-hypertenstives, anti-coagulants (e.g. heparin), belimumab, bisphosphonate derivative; patients who have had or are planning to have the following invasive procedures are not eligible; patients with evidence of active bleeding, known or prior history in prior 3 months of esophageal varices, patients with a history of CNS arterial/venous thromboembolic events including transient ischemic attack (TIA) or cerebrovascular accident (CVA) within 6 months prior to study enrollment are not eligible, patients with a history of deep vein thrombosis (including pulmonary embolism) within 3 months prior to study enrollment are not eligible, patients with a history of hemoptysis or other signs of pulmonary hemorrhage within 3 months prior to study enrollment are not eligible, patients with a history of ≥Grade 3 bleeding disorders, vasculitis, or had a significant (≥Grade 3) episode from gastrointestinal bleeding, within 6 months prior to enrollment are not eligible, patients with CNS tumors and evidence of new CNS hemorrhage of more than punctate size and/or more than three foci of punctate hemorrhage on baseline MRI obtained within 14 days prior to study enrollment are not eligible (ECHO Gradient MRI sequences per institutional guidelines are required for patients with CNS tumors);patients with known cardiac disease per the New York Heart Association definition such as myocardial infarction, severe or unstable angina, peripheral vascular disease, or congestive heart failure or peripheral vascular disease are not eligible; patients who have a history of fistula, gastrointestinal ulcer or perforation, or intra-abdominal abscess within 3 months of study enrollment are not eligible; patients with a history of hypertensive crisis or hypertensive encephalopathy within 6 months of study enrollment are not eligible; patients who have non-healing wound, unhealed or incompletely healed fracture, or a compound (open) bone fracture at the time of enrollment are not eligible; immunocompromised patients (other than that related to the primary oncologic diagnosis or to the use of corticosteroids) including patients known to be HIV positive are not eligible; patients who have an uncontrolled infection are not eligible; patients who have received a prior solid organ transplantation are not eligible; and patients who in the opinion of the investigator may not be able to comply with the safety monitoring requirements of the study are not eligible.

Eligible patients will receive a cycle of therapy over 42 days where ramucirumab is administered i.v. over 60 munuts on Day 1, 15, 29, and disease is evaluated on Day 42. The cycle may be repeated every 42 days if the patient has at least stable disease and has again met the conditions for eligibility. The dose of ramucirumab administered is between about 6 mg/kg and about 16 mg/kg.

Patients should receive diphenhydramine (1 mg/kg, maximum dose 50 mg) (or alternative antihistamine) within 30 to 60 minutes prior to each infusion with ramucirumab. Anaphylactic precautions should be observed during ramucirumab administration. If ≥Grade 2 infusional reaction occurs, the infusion should be stopped and supportive care given as per institutional guidelines.

Patients treated with ramucirumab may have any of the following pediatric cancers: osteosarcoma, sarcoma, pheochromocytoma, paraganglioma, Wilms' tumor, hepatoblastoma, carcinoma NOS (not otherwise specified), synovial sarcoma, and desmoplastic small round cell tumor.

As of Apr. 5, 2018, sixteen patents are enrolled, one patient enrolled on DL1 was ineligible due to administration of a restricted medication (NSAIDS). Said patients included patients diagnosed with osteosarcoma (3), other sarcoma (6), pheochromocytoma/paraganglioma (2), Wilms tumor (1), hepatoblastoma (1), and carcinoma (2). ⅙ patients receiveing a dose of 8 mg/kg had dose limiting proteinuria (grade 2), ⅗ patients achieved Cₘᵢₙₛₛ>50 μg/ml. Initially 6 patients were enrolled to receive a dose of 12 mg/kg, of these ⅙ had DLT (dose limiting toxicities), however, only 4 were evaluable for PK; 2 additional patients were enrolled. Overall, for patients receiving 12 mg/kg, ⅛ had DLT (proteinuria, grade 2), and 6/6 patients achieved target concentration Cₘᵢₙₛₛ≥50 μg/ml. Neither patient with DLT completed PK sampling. Enrollment continues for the 12 mg/kg dose for children <12 y. The median (range) duration of protocol therapy was 1 (1-8) cycle(s). No patient experienced DLT after Cl and toxicity profile in subsequent cycles was similar to adults. Response evaluation (RECIST) is ongoing.

To date, ramucirumab is well tolerated with a toxicity profile consistent with class-effect. No subject experienced grade ≥3 adverse events. Dose limiting proteinuria (Grade 2) occurred in DL 1 and DL 2. However, an MTD was not reached. Using toxicity and target trough drug concentration as endpoints, Ramucirumab 12 mg/kg IV every 2 weeks was identified as the dose for PK expansion. PK expansion continues in patients <12 y. After establishing the RP2D in solid tumors, the safety, tolerability and response to Ramucirumab will be evaluated in children and adolescents with relapsed or refractory Brain Tumors.

SEQUENCE LISTING

SEQ ID NO: 1 (Anti-Human VEGFR-2 Antibody, LCVR) (Artificial Sequence)
DIQMTQSPSSVSASIGDRVTITCRASQGIDNWLGWYQQKPGKAPKWYDASNLD
TGVPSRFSGSGSGTYFTLTISSLQAEDFAVYFCQQAKAFPPTFGGGTKVDIK
SEQ ID NO: 2 (Anti-Human VEGFR-2 Antibody, HCVR) (Artificial Sequence)
EVQLVQSGGGLVKPGGSLRLSCAASGFTFSSYSMNWVRQAPGKGLEWVSSISSSS
SYIYYADSVKGRFTISRDNAKNSLYLQMNSLRAEDTAVYYCARVTDAFDIWGQG
TMVTVSS
SEQ ID NO: 3 (Anti-Human VEGFR-2 Antibody, LC) (Artificial Sequence)
DIQMTQSPSSVSASIGDRVTITCRASQGIDNWLGWYQQKPGKAPKWYDASNLD
TGVPSRFSGSGSGTYFTLTISSLQAEDFAVYFCQQAKAFPPTFGGGTKVDIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 4 (Anti-Human VEGFR-2 Antibody, HC) (Artificial Sequence)
EVQLVQSGGGLVKPGGSLRLSCAASGFTFSSYSMNWVRQAPGKGLEWVSSISSSS
SYIYYADSVKGRFTISRDNAKNSLYLQMNSLRAEDTAVYYCARVTDAFDIWGQG
TMVTVSSASTKGPSVLPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYT
QKSLSLSPGK
SEQ ID NO: 5 (Human VEGFR-2) (Homo Sapiens)
MQSKVLLAVALWLCVETRAASVGLPSVSLDLPRLSIQKDILTIKANTTLQITCRGQ
RDLDWLWPNNQSGSEQRVEVTECSDGLFCKTLTIPKVIGNDTGAYKCFYRETDL
ASVIYVYVQDYRSPFIASVSDQHGVVYITENKNKTVVIPCLGSISNLNVSLCARYP
EKRFVPDGNRISWDSKKGFTIPSYMISYAGMVFCEAKINDESYQSIMYIVVVVGY
RIYDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKPFV
AFGSGMESLVEATVGERVRIPAKYLGYPPPEIKWYKNGIPLESNHTIKAGHVLTEVI
EVSERDTGNYTVILTNPISKEKQSHVVSLVVYVPPQIGEKSLISPVDSYQYGTTQT
LTCTVYAIPPPHHIHWYWQLEEECANEPSQAVSVTNPYPCEEWRSVEDFQGGNKI
EVNKNQFALIEGKNKTVSTLVIQAANVSALYKCEAVNKVGRGERVISFHVTRGPE
ITLQPDMQPTEQESVSLWCTADRSTFENLTWYKLGPQPLPIHVGELPTPVCKNLD
TLWKLNATMFSNSTNDILEVIELKNASLQDQGDYVCLAQDRKTKKRHCVVRQLT
VLERVAPTITGNLENQTTSIGESIEVSCTASGNPPPQIMWFKDNETLVEDSGIVLKD
GNRNLTIRRVRKEDEGLYTCQACSVLGCAKVEAFFIIEGAQEKTNLEIIILVGTAVI
AMFFWLLLVIILRTVKRANGGELKTGYLSIVMDPDELPLDEHCERLPYDASKWEF
PRDRLKLGKPLGRGAFGQVIEADAFGIDKTATCRTVAVKMLKEGATHSEHRALM
SELKILIHIGHHLNVVNLLGACTKPGGPLMVIVEFCKFGNLSTYLRSKRNEFVPYK
TKGARFRQGKDYVGAIPVDLKRRLDSITSSQSSASSGFVEEKSLSDVEEEEAPEDL
YKDFLTLEHLICYSFQVAKGMEFLASRKCIHRDLAARNILLSEKNVVKICDFGLA
RDIYKDPDYVRKGDARLPLKWMAPETIFDRVYTIQSDVWSFGVLLWEIFSLGASP
YPGVKIDEEFCRRLKEGTRMRAPDYTTPEMYQTMLDCWHGEPSQRPTFSELVEH
LGNLLQANAQQDGKDYIVLPISETLSMEEDSGLSLPTSPVSCMEEEEVCDPKFHY
DNTAGISQYLQNSKRKSRPVSVKTFEDIPLEEPEVKVIPDDNQTDSGMVLASEELK
TLEDRTKLSPSFGGMVPSKSRESVASEGSNQTSGYQSGYHSDDTDTTVYSSEEAE
LLKLIEIGVQTGSTAQILQPDSGTTLSSPPV

1. A method of treating cancer in a human patient comprising administering a therapeutically effective amount of ramucirumab to the human patient in need thereof; wherein the human patient has been diagnosed with one of the following cancers: osteosarcoma, sarcoma, pheochromocytoma, paraganglioma, Wilms' tumor, hepatoblastoma, carcinoma NOS, synovial sarcoma, or desmoplastic small round cell tumor.

2. The method of claim 1, wherein the human patient is at least twelve months of age, but less than or equal to twenty one years of age.

3. The method of claim 1, wherein the cancer is osteosarcoma.

4. The method of claim 1, wherein the cancer is sarcoma.

5. The method of claim 1, wherein the cancer is pheochromocytoma.

6. The method of claim 1, wherein the cancer is paraganglioma.

7. The method of claim 1, wherein the cancer is Wilms' tumor.

8. The method of claim 1, wherein the cancer is hepatoblastoma.

9. The method of claim 1, wherein the cancer is carcinoma NOS.

10. The method of claim 1, wherein the cancer is synovial sarcoma.

11. The method of claim 1, wherein the cancer is desmoplastic small round cell tumor.

12. The method of claim 1, wherein ramucirumab is administered intravenously every 2 weeks at a dose from about 6 mg/kg to about 16 mg/kg.

13. The method of claim 1, wherein ramucirumab is administered intravenously every 2 weeks at a dose from 6 mg/kg to 16 mg/kg.

14. The method of claim 1, wherein ramucirumab is administered intravenously every 2 weeks at a dose from about 8 mg/kg to about 12 mg/kg.

15. The method of claim 1, wherein ramucirumab is administered intravenously every 2 weeks at a dose from 8 mg/kg to 12 mg/kg.

16. The method of claim 1, wherein ramucirumab is administered intravenously every 2 weeks at a dose of about 6 mg/kg.

17. The method of claim 1, wherein ramucirumab is administered intravenously every 2 weeks at a dose of about 8 mg/kg.

18. The method of claim 1, wherein ramucirumab is administered intravenously every 2 weeks at a dose of about 10 mg/kg.

19. The method of claim 1, wherein ramucirumab is administered intravenously every 2 weeks at a dose of about 12 mg/kg.

20. The method of claim 1, wherein ramucirumab is administered intravenously every 2 weeks at a dose of about 16 mg/kg.

21-56. (canceled)


SEQUENCE LISTING (5 sequences)

SEQ ID NO: 1 - protein, 107 aa, Artificial Sequence [sequence omitted]

SEQ ID NO: 2 - protein, 116 aa, Artificial Sequence [sequence omitted]

SEQ ID NO: 3 - protein, 214 aa, Artificial Sequence
  Synthetic Construct
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Artificial Sequence
  Synthetic Construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV